(19) United States
(12) Patent Application Publication
(10) Pub. No.: US 2025/0040502 A1
(43) Pub. Date: Feb. 6, 2025

(54) METHOD OF PRESERVING MAIZE POLLEN VIABILITY UNDER HEAT STRESS

(71) Applicant: SYNGENTA CROP PROTECTION AG, Basel (CH)

(72) Inventors: Nathinee RUTA, Basel (CH); Antoine FONTAN, Lombez (FR); Angelique NICOLI, Lombez (FR); Joerg LEIPNER, Stein (CH)

(73) Assignee: SYNGENTA CROP PROTECTION AG, Basel (CH)

(21) Appl. No.: 18/715,432

(22) PCT Filed: Dec. 1, 2022
(86) PCT No.: PCT/EP2022/084016

§ 371 Date: May 31, 2024

(30) Foreign Application Priority Data
Dec. 2, 2021 (EP) 21212038.0

(51) Int. Cl. A01H 3/00; A01G 22/20; A01N 3/00

(52) U.S. Cl. CPC A01H 3/00; A01G 22/20; A01N 3/00

(57) ABSTRACT

A method of preserving the pollen viability of maize, especially under heat stress, comprising treating the maize plants with a protein hydrolysate.

The present invention relates to a method of improving pollen viability in maize plants, especially under heat stress, using a protein hydrolysate.

Maize (Zea mays), also known as corn, is one of the most widely cultivated crops in global agriculture. It is grown for human consumption, animal feed, com ethanol and other corn products such as corn starch and corn syrup.

Protein hydrolysates are an emerging class of crop management products utilized in agriculture for improving nutrient assimilation and mitigating crop stress. Protein hydrolysates are made by the acid or base hydrolysis of plant or animal proteins. The hydrolysate is generally a mixture of free amino acids, short peptides, and long peptides, depending on hydrolysis conditions. They are known to have certain biostimulant effects when applied to plants. A review of some of these is given in Front. Plant Sci., 22 Dec. 2017 accessible at https://doi.org/10.3389/fpls.2017.02202.

Pollen viability is the ability of the pollen to transfer the male gametes produced by them to the embryo sac for pollination and it can be measured by membrane integrity using an impedance flow cytometer (brand Amphasys Z32 Pollen Analyzer). Successful pollination requires viable pollen; however, the viability of pollen can be negatively impacted through external factors such as heat stress or drought stress.

We have now found that protein hydrolysates when applied to maize have the surprising effect of improving pollen viability under heat stress, an effect that has not been previously reported.

According to the present invention, there is provided a method of improving the pollen viability of a maize plant comprising treating a maize plant or the locus thereof with a protein hydrolysate. Preferably the method is carried out on a maize plant that is subsequently subject to heat stress.

According to the present invention there is provided a process of applying a protein hydrolysate to a maize plant or the locus thereof for the purpose of improving pollen viability. Preferably the process is carried out for the purpose of improving pollen viability under heat stress. There is also provided the use of a protein hydrolysate for preserving the pollen viability of a maize plant.

Preferably the protein hydrolysate is from an animal source, such as collogen. Many protein hydrolysates are commercially available, and a particularly preferred protein lysate is ISABION (CAS number 9015-54-7; EC number 310-296-6), available from Syngenta Crop Protection AG. It is in the form of a suspension concentrate of 100 g/l total amino acids in water. ISABION comprises free amino acids (11%) and peptides (52%) which together make 62.5% of total amino acids.

Protein hydrolysate can be used in unmodified form or together with adjuvants conventionally employed in the art of formulation. They are generally supplied in the form of suspension concentrates which are diluted with water before use. Typically, the concentrates contain between 50 and 250 g/l of protein hydrolysate based on total amino acid content. Preferably these concentrates are diluted with water before use by a factor of 10 to 50 so that the final spray solution comprises between around 0.1 to 1 g/l of protein hydrolysate, preferably 0.2 to 0.5 g/l, based on total amino acid content.

Heat stress means conditions in which the average ambient temperatures where the maize is growing are significantly above those normally expected, for example more than 5° C., especially more than 8° C. above temperatures normally expected. Examples of heat stress conditions are daytime temperatures above 30° C., especially above 35° C. and nighttime temperatures above 20° C., especially above 23° C.

Application of the protein hydrolysate can be by conventional agricultural spray. Typical application rates of the protein hydrolysate are 0.5 to 5 litre/ha, more typically 1 to 2 litre/ha of final spray solution.

The protein hydrolysate can be applied simultaneously along with other agrochemicals or fertilisers, for example maize-selective herbicides, fungicides, or insecticides.

There are two main growth stages of corn, the vegetative stages (V) and the reproductive stages (R).

TABLE 1
Corn vegetative stages:

| Vegetative Stage | Description & Time After Planting* |
|---|---|
| VE (Emergence) | Can occur four to five days after planting under ideal conditions, but up to two |
| | weeks or longer under cool or dry conditions. |
| V1-V5 | At V1, first collared leaf appears with round tip; nodal roots develop. The |
| | number of collared leaves increases By V2, plant is 2 to 4 inches tall and |
| | relies on the energy in the seed. Number of kernel rows being determined. |
| | Nodal roots taking over the seminal root system. |
| V6-V8 | Growing at or just above soil surface. Ear shoots and tillers may be visible. |
| | New collared leaf every 4 to 5 days. Lower leaves may no longer be visible. |
| V9-V11 | Around six to eight weeks after VE, rapid stalk internode elongation. Tassel |
| | is about to begin development. Rapid root development |
| V12-Vnth | Number of kernels per row being determined between V12 and V17. Rapid |
| | stalk elongation continues Steady increase in nutrient and dry weight |
| | accumulation (starting from V10). New leaf stage every 2 to 3 days. |
| VT | Tassel Emergence - Tassel extends above last leaf. Last branch of the tassel |
| | is visible. Full plant height is attained. Pollen shed (anthesis) begins hortly |
| | after the corn tassel is fully emerged from the whorl. Spikelets near the |
| | main axis of the tassel are the first to open, exposing the anthers that bear |
| | pollen grains. About 2 to 3 days from silk emergence (R1) |

*Represents normal corn growth based on favorable temperature and moisture. Cooler temperatures can slow growth while warmer temperatures can increase growth rate and reduce time between leaf stages.

In one embodiment, there is provided a method of preserving the pollen viability of a maize plant comprising, treating the maize plant or the locus thereof, with a protein hydrolysate. There is further provided a method wherein the maize plant is subjected to heat stress. In one embodiment, the heat stress is a daytime temperature above 30° C., and preferably above 35° C. In one embodiment, the heat stress is a daytime temperature above 40° C.

In another embodiment, the heat stress is a night-time temperature above 20° C., and preferably above 23° C. In one embodiment, the heat stress is a night-time temperature above 26° C.

In one embodiment, the protein hydrolysate is applied to a maize plant or the locus thereof, during the vegetative stage of growth. Preferably, the protein hydrolysate is applied to a maize plant or the locus thereof, during vegetative growth stages V9-V11.

Maize includes varieties includes conventional varieties as well as those that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. Examples of maize that has been rendered tolerant to herbicides or classes of herbicides by genetic engineering methods include glyphosate- and glufosinate-resistant maize varieties commercially available under the trade names RoundupReady®, Herculex I® and LibertyLink®.

Maize also includes those varieties which have been transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA (b) toxin); YieldGard Rootworm® (maize variety that expresses a CryIIIB (b1) toxin); YieldGard Plus® (maize variety that expresses a CryIA (b) and a CryIIIB (b1) toxin); Starlink® (maize variety that expresses a Cry9 (c) toxin); Herculex I® (maize variety that expresses a CryIF (a2) toxin and the enzyme phosphinothricine N-acetyltransferase (PAT) to achieve tolerance to the herbicide glufosinate ammonium); Agrisure® CB Advantage (Bt11 corn borer (CB) trait), Agrisure® RW (corn rootworm trait) and Protecta®.

Maize also includes varieties which have been transformed by the use of recombinant DNA techniques so that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by d-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins, and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic maize capable of synthesising such toxins are disclosed, for example, in EP-A-0 374 753, WO93/07278, WO95/34656, EP-A-0 427 529, EP-A-451 878 and WO 03/052073.

The toxin contained in the transgenic plants imparts to the plants tolerance to harmful insects. Such insects can occur in any taxonomic group of insects but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NatureGard®, Agrisure® GT Advantage (GA21 glyphosate-tolerant trait), Agrisure® CB Advantage (Bt11 corn borer (CB) trait) and Protecta®.

Further examples of such transgenic crops are Bt11 Maize from Syngenta, Bt176 Maize from Syngenta, MIR604 Maize from Syngenta, MON 863 Maize from Monsanto, 1507 Maize from Pioneer, NK603×MON 810 Maize from Monsanto.

### EXAMPLES

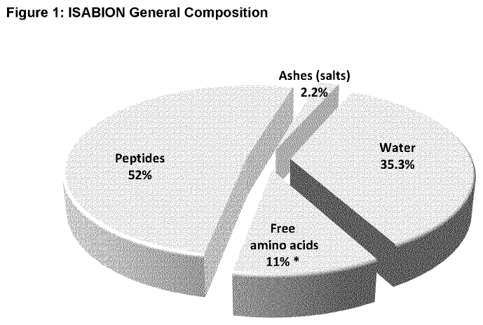

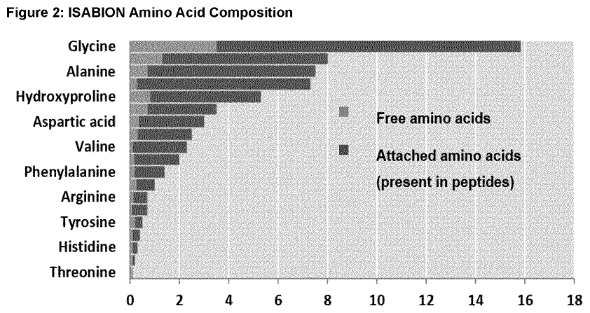

The Examples which follow serve to illustrate the invention. The examples use a commercial protein hydrolysate ISABION available from Syngenta Crop Protection AG. ISABION has the technical characteristics shown in FIGS. 1 (ISABION General Composition), 2 (ISABION Amino Acid Composition), and 3 (ISABION Hydrolysis).

### 1. Method

ISABION was applied to five different varieties of maize plants ANA1416, CNA1124, AA2359, DAX3360 and ITPJ8713. The ISABION was applied by spray application at 2 rates corresponding to 1 litre of ISABION per hectare and 2 litres of ISABION per hectare with 200L/ha spray volume and otherwise identical untreated maize plants were used as a standard for comparison. The application timing is between vegetative growth stages V9-11. Three replicates were carried out for each application. At anthesis (pollen shed-VT stage), 5 tassels were randomly selected per replicate and pollen viability was measured using an Amphasis Z32 Pollen Analyzer as DO (after field sampling). The tassels then divided into 2 modelities, one as “D24” without stress under conditions 28° C./18° C. (daytime/night-time temperature), 60% RH (relative humidity) with 14 hour photoperiod. Another one as “D24 STRESS” for heat stress treatment in the growth chamber. Heat stress conditions were set at 40° C./26° C. (daytime/night-time temperature), 60% RH (relative humidity) with 14 hour photoperiod. Pollen viability from corn subject to these two modelities were then measured.

### 2. Results

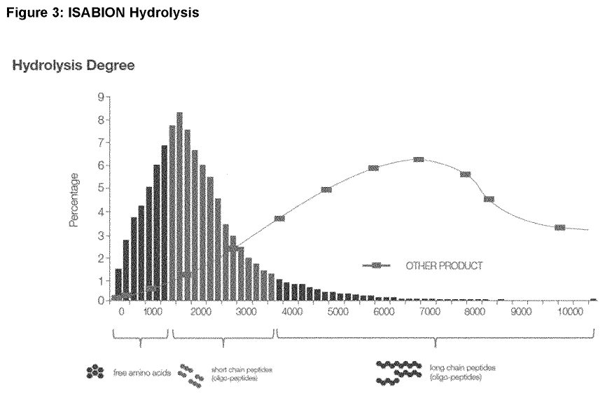

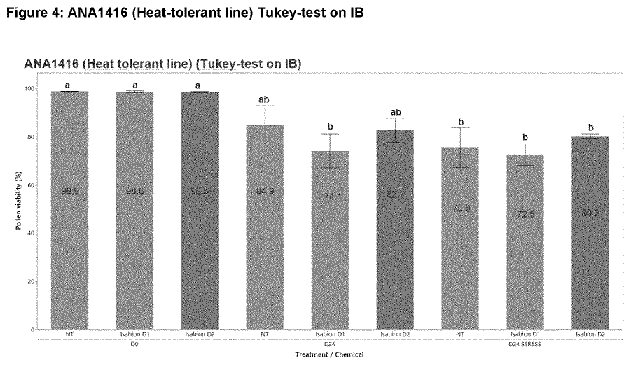

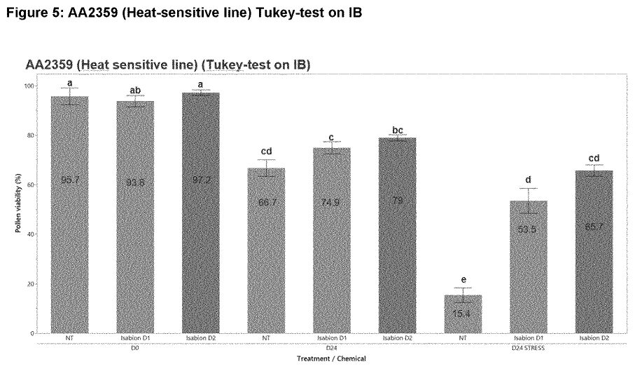

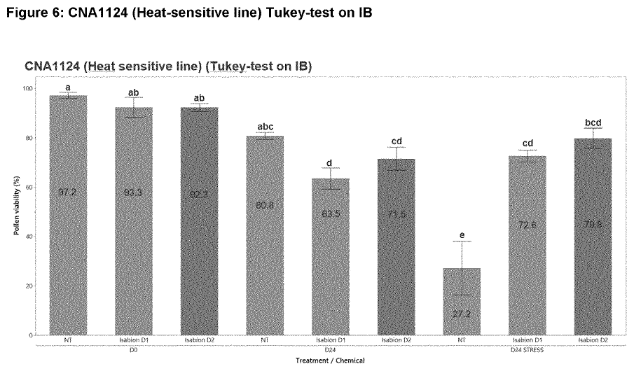

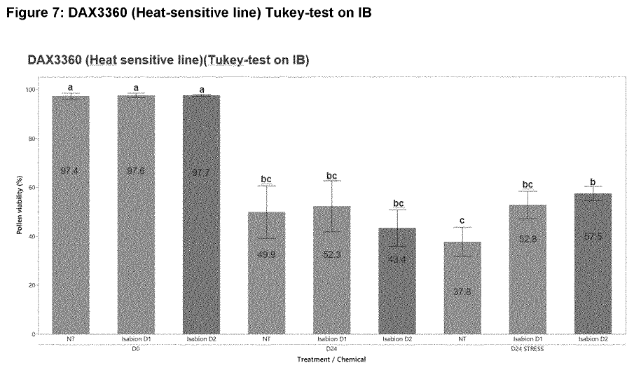

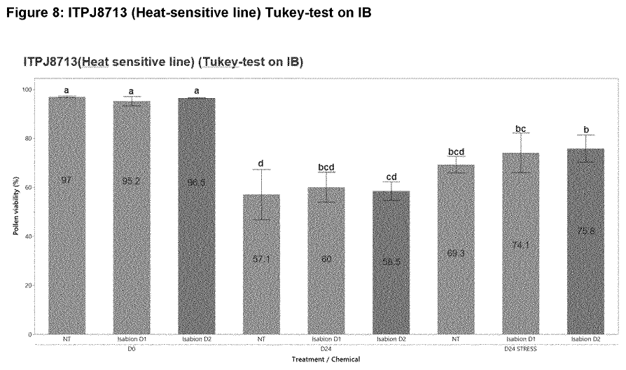

The results clearly show that ISABION improves pollen viability in these tests. For a heat tolerant corn line ANA1416 no significant difference between any treatments under D24 and D24 STRESS conditions (see FIG. 4) were observed. While for early variety, heat tolerant lines AA2359 (FIG. 5), CNA1124 (FIG. 6) and DAX3360 (FIG. 7) treatments of ISABION at 1L/ha (D1) and 2L/ha (D2) showed significant higher percentage of pollen viability compared to untreated (NT) under D24 STRESS, lower effects of heat stress were observed with the late variety, heat tolerant line ITPJ8713 (FIG. 8) where D24 STRESS showed higher pollen viability than D24 (non-heat stress). This may be due to the impact of weather conditions on late variety. However, a trend of increase of pollen viability was observed with the two treatments of ISABION compared with non-treated (NT).

1. A method of preserving the pollen viability of maize comprising, treating the maize plants or the locus at which the maize plants are growing, with a protein hydrolysate.

2. A method according to claim 1 carried out on maize that is subsequently subject to heat stress.

3. A method according to claim 2, wherein the heat stress is a daytime temperature above 30° C.

4. A method according to claim 2, wherein the heat stress is a night-time temperature above 20° C.

5. A process of applying a protein hydrolysate to a maize plant or the locus thereof for the purpose of improving pollen viability.

6. A process according to claim 5 carried out for the purpose of improving pollen viability under heat stress.

7. A method according to claim 1, wherein the protein hydrolysate is applied to the maize plants or the locus at which the maize plants are growing, during the vegetative stage of growth.

8. A method according to claim 1 in which the protein hydrolysate is from an animal source.

9. A method according to claim 1 in which the protein hydrolysate is ISABION.

10. A method according to claim 1 in which the protein hydrolysate is applied at a rate of 0.5 to 5 litres per hectare.

11. Use of a protein hydrolysate for preserving the pollen viability of maize.
